(19) United States
(12) Patent Application Publication
(10) Pub. No.: US 2011/0020228 A1
(43) Pub. Date: Jan. 27, 2011

(54) TARGETED CELLULAR SELECTIVITY OF SURFACE ACTIVE MOLECULES

(75) Inventors: Brij M. Moudgil, Gainesville, FL (US); Manoj Varshney, Gainesville, FL (US); Stephen R. Grobmyer, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: University of Florida Resarch Foundation, Inc., Gainesville, FL (US)

(21) Appl. No.: 12/865,589

(22) PCT Filed: Feb. 2, 2009
(86) PCT No.: PCT/US09/32851

§ 371 Date: Oct. 11, 2010

Related U.S. Application Data
(60) Provisional application No. 61/063,196, filed on Feb. 1, 2008.

(51) Int. Cl. A61K 49/00; A61K 31/765; A61K 31/20; A61K 31/201; A61K 31/23; A61K 31/231; A61K 36/47; A61K 36/889; A61K 36/899; A61K 31/341; A61K 31/08; A61K 31/355; A61K 31/7024; A61K 31/05; A61K 31/575; A61K 31/221; A61K 31/255; A61P 35/00

(52) U.S. Cl. 424/9.1; 424/78.31; 514/558; 514/560; 514/552; 424/731; 424/727; 424/750; 514/473; 424/78.38; 514/722; 514/458; 514/23; 514/731; 514/182; 514/551; 514/517

(57) ABSTRACT

A method for the treatment of cancer involves delivering a surface active agent to an organism, where the surface active agent selectively partitions to and kills cancer cells as opposed to healthy cells. The surface active agent can be an ionic or a non-ionic surfactant with a HLB of less than 29 or a mixture of surface active agents with a HLB of less than 40, where the hydrophobic portion is a lesser fraction of the surface active agent than the hydrophilic portion. A fluorescence method of detecting and locating cancer cells in an organism involves delivering a surface active agent, where the surface active agent includes a fluorescence moiety that upon selective partitioning of the surface active agent to the cancer cells and irradiation by a radiation source to excite the fluorescence moiety, a fluorescence emission is observed permitting the detection and location of the cancerous tissue by local volumes of relatively high intensity emission.

### CROSS-REFERENCE TO RELATED APPLICATION

The present application claims the benefit of U.S. Provisional Application Ser. No. 61/063,196, filed Feb. 1, 2008, which is hereby incorporated by reference herein in its entirety, including any figures, tables, or drawings.

### GOVERNMENT SUPPORT

The subject matter of this application has been supported by a research grant from the National Institutes of Health under grant number RR020654-01. Accordingly, the government has certain rights in this invention.

### BACKGROUND OF THE INVENTION

Cancer is the second leading cause of death globally. At an early stage, when cancer is restricted to a local area of the body, it can be cured by surgical operation or by ionizing radiation, however, once dissemination of cancer has taken place, chemotherapy, radiotherapy, and immunotherapy, or their combinations, become the main treatment procedures. Chemotherapy is a complicated procedure in which many factors are involved in determining its success or failure. It carries a high risk as it kills normal cells along with cancer cells, and the more effective drugs tend to be more toxic to normal cells. Problems may still exist even after successful chemotherapy, and the patients have to tolerate severe side effects and sacrifice their quality of life. Thus, new methods and techniques for killing cancer cells selectively with minimal side effects to normal cells are of utmost need.

Recent trends in colloids and surface science are to use micelles, microemulsions, and emulsions for encapsulating insoluble cancer drugs. Many surfactants are known that are non-toxic or display a very low toxicity. A number of studies have examined the effect of the inclusion of a nonionic surfactant on the efficacy of the drug during treatment. Little effort has been directed to the use of nonionic surfactants free of drugs for attacking cancer cells. However, Pluronic® non-ionic surfactants, polyethylene-block-polypropylene-block-polyethylenes (PEO-block-PPO-block-PEO) have been examined as potential cancer preventing agents.

Silk et al. (Cancer, 1972, January, 171-2) concluded that Pluronic® F68 was effective in suppressing the development of tumor metastasis in rats injected with 100,000 cells of Walker 256 ascitic tumor, where F68 was provided at 4 mg/100 g body weight intravenously per day for 7 days prior and 7 days following the injection of the cells. Metastasis occurred in 16% of the rats treated with F68 as opposed to 85% of a similar sized control group over a period of 8 weeks following injection of the cells.

Parnaud et al. (British Journal of Cancer, 2001, 84(1) 90-3) examined the prevention of colorectal cancer, induced by injections of axoxymethane, by Pluronic® F68, Pluronic® F127, Pluronic® F85, Pluronic® F64, Pluronic® F61 and PEG 8000, a polyethylene glycol of 8000 molecular weight known as a suppressor of aberrant crypt foci in rats, when these polyethers were included in the diet of the rats. Again, Pluronic®F68 was found to be very effective as a suppressor of carcinogenesis, displaying results superior to that of PEG 8000, yet none of the other pluronics displayed results superior to the control. Pluronic® F68 has a HLB (hydrophilic-lipophilic balance) value of 29, and has two PEO blocks of 82 ethylene oxide repeating units and a PPO block with 31 propylene oxide repeating units.

Only Pluronic® F68 has demonstrated any efficacy without a drug with regard to the prevention of cancer. However, this surfactant was administered prior to introduction of cancer cells or an inducer of cancer cells to a host, but its ability to treat an established malignancy was not addressed. The mechanism by which prevention occurs was not addressed and the utility of F68 for the treatment of an existing malignancy was not disclosed.

Of the studies where PEO-block-PPO-block-PEO has been included with a drug for cancer treatment, the advantage appears to be in the hypersensitization of multiple drug resistant (MDR) cells. Bartrkova et al. (British Journal of Cancer, 2001, 88, 12, 1987-97) indicates that the sensitization effect of the copolymer appears to be related to energy depletion (ATP depletion). An earlier study, Bartrkova et al. (Pharmaceutical Research 1999, 16, 1373-9) indicates that drug uptake is enhanced by the pluronic copolymers which inhibit P-glycoprotein efflux systems and the sequestering into cytoplasmic compartments of the MDR cells, which are two phenomena that are ATP dependent. It was concluded that the appropriate pluronics for maximum potency are those with large sized PPO blocks and short PEO blocks.

Because of their low toxicity, surfactants would be desirable therapeutic agents relative to nearly all existing methods of chemotherapy, where low toxicity agents have little or no specificity for malignant cells over healthy cells. Hence the goal of identifying surfactants that act as agents for the treatment of an existing cancer remains.

### BRIEF SUMMARY OF THE INVENTION

The subject invention is directed to a method of treating cancer where a surface active molecule is delivered to an organism having cancer. In one embodiment, the surface active molecule has a HLB of less than about 29, and selectively partitions to cancer cells rather than healthy cells such that primarily cancer cells are attacked and killed by the surface active molecule. In another embodiment, a mixture of surface active molecules with different HBL values where the combined HBL is less than 40 can be used in place of a single surface active molecule.

In one embodiment of the invention the surface active molecule is a non-ionic surfactant. The non-ionic surfactant is one with a relatively high hydrophilic fraction to the hydrophobic fraction, but with a sufficient hydrophobic fraction to disrupt the cancer cell membrane. An exemplary non-ionic surfactant is a polyoxyethylene-polyoxypropylene-polyoxyethylene tri-block copolymer where the polyethylene blocks are of a higher degree of polymerization as the polypropylene block. In an alternate embodiment of the invention, the surface active agent can be an ionic surfactant.

The surface active molecule can be injected intravenous, intra-arterial, intradermal, intraperitoneal, intramuscular, subcutaneous, or into any other tissue as a mode of delivery. Alternately, delivery can be carried out by applying the surface active molecule topically, by inhaling, or by oral ingesting. The treatment can be carried in a series of doses or can be carried out continuously. Portable delivery devices such as a pump can be used for administration of the surface active molecule.

The invention is also directed to a method of detection and location of cancer cells and tissue by providing a surface active molecule that includes a fluorescence moiety, has a HLB of less than about 29, and selectively partitions primarily to cancer cells over healthy cells. In this embodiment, the surface active molecule, having concentrated with the cancer cells, is irradiated with electromagnetic radiation to excite the fluorescence moiety so that a relatively high fluorescence emission from concentrated surface active molecules indicates the presence of cancer cells, and can be used to map the location of the cancer cells.

### BRIEF DESCRIPTION OF THE DRAWINGS

FIG. 1 shows a graph of the effect of L61 and L63 on A549 Cancer Cells and human normal RBC. Results are mean±S.E.M. (n=6).

FIG. 2 shows a graph of the effect of F77 and F127 on A549 Cancer Cells and human normal RBC. Results are mean±S.E.M. (n=6).

FIG. 3 shows a graph of the effects of L63 (8 mM) dose on Human Red Blood Cells. Results are mean±S.E.M. (n=6).

FIG. 4 shows a graph of the effects of F127 (8 mM) dose on. Human Red Blood

Cells. Results are mean±S.E.M. (n=6).

FIGS. 5A-5C are reproductions of light micrographs illustrating the effect of Pluronic F77 dose on A549 cancer cells.

FIG. 6 shows a graph of the selective effect of F77 on BT474 Cancer Cells over normal epithelial cells.

### DETAILED DESCRIPTION OF THE INVENTION

Cancer cell membranes are often less hydrophobic than a normal cell membrane because of a lack of cholesterol, which is a hydrophobic molecule. Higher fluidity of plasma membranes is a characteristic of most cancer cells that results from the lower concentration of cholesterol in cancer cells. In addition, tumor blood vessels are lined with an over-abundance of negative charge particles and cancer cells have a lower degree of intercellular adhesion than do healthy cells. The invention provides methods of treating and/or detecting cancer cells. The methods of the subject invention exploit the difference in hydrophobicity of cancer cells and healthy cells. Specifically, the method of the subject invention uses surface active molecules that have a greater affinity for the more hydrophilic cancer cells. The surface active molecules, having a relatively high hydrophilic fraction and a relatively low hydrophobic fraction, are selected for entry into cancer cells by an endocytotic active mechanism, diffusing into polar regions of the cancer cell and disrupting the cell membrane.

One embodiment of the invention is directed to a method of treating cancer with a surfactant that selectively partitions to cancer cells rather than normal cells. A specific surfactant can be active towards a specific cancer cell or be active towards a variety of cancer cells. The surfactant, alone, is able to attack the cancer cells without the inclusion of a highly toxic anti-cancer drug. Among the cell potentially treated with the surfactants are those of breast, prostate, colon, CNS, ovarian, renal, liver, pancreatic, uterine, or lung tumors as well as human leukemia or melanoma cells.

The subject invention further provides methods of use of the surfactants for inhibiting tumors and other cancer cells in an animal, preferably a mammal. The invention comprises a method for the antitumor treatment of a human in need of such treatment, such as a human hosting cancer cells, including breast, prostate, colon, CNS, ovarian, renal, liver, pancreatic, uterine, or lung tumors as well as human leukemia or melanoma cells.

In another embodiment of the invention, surfactants can contain specific groups that permit the targeting or detection of a cancer cell. For example, a surfactant selective for cancer cells can be modified to include a fluorescent or phosphorescent dye such that the aggregation of the dye modified surfactant can indicate the presence and/or location of cancer cells. The primary mode for selectivity of the surfactant for a cancer cell results from the different cell wall structure of cancer cells and healthy cells.

The dosage administration of the surfactant to a host will depend on the identity of the cancer cells, the type of host involved, its age, weight, health, type of other concurrent treatment, if any, frequency of treatment, and therapeutic ratio. Formulation of the dosage form can be carried out according to known methods for preparing pharmaceutically useful compositions. The surfactants can be combined in an effective amount with a suitable carrier to facilitate effective administration of the surfactant.

In one embodiment of the invention, the surfactant is polyoxyethylene-polyoxypropylene-polyoxyethylene tri-block copolymers or PEO-block-PPO-block-PEO where the absolute and relative sizes of the PEO and PPO blocks can be optimized to selectively target cancer cells. PEO homopolymers are highly structurally regular and highly water soluble and are considered non-toxic. PPO is generally an atactic polymer with low water solubility.

In another embodiment of the invention, the surfactant for selective partitioning to cancer cells is a non-ionic surfactant such as: polyoxyethylene sorbitol esters; polyethylene glycol stearates; and mixtures of monosterate and distearate esters of mixed macrogols (polyoxyethylene polymer) and free glycol, such as macrogol 15 hydroxystearate available commercially as Solutol® HS 15 from BASF Aktiengesellschaft.

In another embodiment of the invention, the surfactant for selective partitioning to cancer cells is a non-ionic surfactants such as: alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction products of polyols and at least one member of the group consisting of fatty acids, glycerides, vegetable oils, and hydrogenated vegetable oils; sugar esters; sugar ethers; and sucroglycerides.

In another embodiment of the invention the surfactant for selective partitioning to cancer cells is a non-ionic hydrophilic surfactant derived from a reaction product of a polyol (glycerol, ethylene glycol, polyethylene glycol, sorbitol, propylene glycol, pentaerythritol, a saccharide) and monoglyceride, diglyceride, triglyceride, or a mixture thereof.

In another embodiment of the invention the surfactant for selective partitioning to cancer cells is a non-ionic hydrophilic surfactant such as PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG40 stearate, PEG-100 stearate, PEG-20 dilaurate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate monoglycerides, PEG-6 caprate/caprylate diglycerides, PEG-8 caprate/caprylate monoglycerides, PEG-8 caprate/caprylate diglycerides, polyglyceryl-10 laurate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, polyglyceryl-10 oleate, Tween 40, Tween 60, sucrose monostearate, sucrose monolaurate, sucrose monopalmitate, PEG 10-100 nonyl phenol series, and PEG 15-100 octyl phenol series.

In another embodiment of the invention the surfactant for selective partitioning to cancer cells is an ionic hydrophilic surfactant, such as: bile acids and salts, analogues, and derivatives thereof; carntine fatty acid ester salts; salts of alkylsulfates; salts of fatty acids; sodium docusate; acyl lactylates; mono-acetylated tartaric esters of mono- and diglycerides, diacetylated tartaric acid esters of mono- and diglycerides; succinylated monoglycerides; and citric acid esters of mono- and diglycerides.

In another embodiment of the invention, the surfactant for selective partitioning to cancer cells is an ionic hydrophilic surfactant such as: lactylic esters of fatty acids; stearoyl-2-lactylate; stearoyl lactylate; succinylated monoglycerides; mono-acetylated tartaric esters of mono- and diglycerides; diacetylated tartaric acid esters of mono- and diglycerides; citric acid esters of mono- and diglycerides; cholate; taurocholate; glycocholate; deoxycholate; taurodeoxycholate; chenodeoxycholate; glycodeoxycholate; glycochenodeoxycholate; taurochenodeoxycholate; ursodeoxycholate; lithocholate; tauroursodeoxycholate; glycoursodeoxycholate; cholylsarcosine; N-methyl taurocholate; caproate; caprylate; caprate; laurate; myristate; palmitate; oleate; ricinoleate; linoleate; linolenate; stearate; lauryl sulfate; tetraacetyl sulfate; docusate; lauroyl carnitine; palmitoyl carnitine; and myristoyl carnitine.

In another embodiment of the invention, the surface active agent can be a silicone surfactant. In this embodiment a hydrophobic polysiloxane chain is coupled with a hydrophilic group, for example, a block copolymer of polyethylene and polydimethylsiloxane is the surface active agent.

The treatment method can employ any of a variety of methods to deliver the surface active agent to the cancer cell environment including: intravenous and intra-arterial methods; intradermal methods; injection directly into tissue; intraperitoneal methods; inhalation methods; intramuscular methods, topical methods; subcutaneous methods and oral methods. The methods can be for individual dosing methods or continuous delivery methods, including portable methods. The treatment can be either systemic, regional, or intralesional depending upon the type and severity of the cancer, as well as the accessibility of the cancer cell site.

In another embodiment of the invention, the surfactant contains a fluorescence dye or other fluorescence moiety such that the selective concentration of the dye into the malignant tissue can occur and subsequently be observed by the emission of the light from the malignant tissue after irradiation, for the detection of the presence of cancer cells and to detect the position of the cancer cells in the organism. In this embodiment a surface active molecule selected from those disclosed above for the cancer therapy embodiments, can be modified with any of the following fluorescent molecules. The fluorescent moiety can be derived from: chlorin e6 and its derivative chlorin e6-Cholin e6-ethylenediamide; polyvinylpyrrolidone (Ce6-PVP); N-acetyl-3,7-dihydroxyphen-oxazine and its derivatives; calcein, AM (Glycine, N,N′-[[3′,6′-bis(acetyloxy)-3-oxospiro[isobenzofuran-1(3H), 9′-[9H]xanthene]-4′,5′-diyl]bis(methylene)]-bis[N-[2-acetyloxy)methyoxy]-2-oxoethyl], bis[(acetyloxy)methyl]ester) and its derivates; indocyanine green (ICG) dye and its derivatives; 5-(and -6)-Carboxy-2′,7′-dichlorofluorescein; 5-FAM; 6-Carboxyrhodamine 6G; aminocoumarin or rhodamine sulfonated derivatives (e.g.Alexa); 2′-7′-bis(carboxyethyl)-5(6)-carboxyfluorescein derivatives (e.g. BCECF); 4,4-difluoro-3a,4adiaza-s-indacene derivatives (e.g BODIPY FL); Calcein; carboxyfluorescein diacetate (e.g. CFDA); CI-NERF; DTAF; eGFP; eYFP; FDA; FITC; FlAsH; N-Ethoxycarbonylmethyl-6-methoxyquinolinium, bromide and derivatives (Fluo3, Fluo4 etc); Fluorescein and derivatives (e.g. FITC); Fluoro-Emerald; FM 1-43; Magnesium Green; mHoneydew; MitoTracker Green; NeuroTrace 500/525, green fluorescent Nissl stain-RNA; Nissl; Oregon Green 488; PicoGreen dsDNA quantitation reagent; Rhodamine; Sodium Green Na+; SYBR Green I; SYTO 13-DNA; TO-PRO-1; and TOTO-1-DNA.

### Materials and Methods

Studies were carried out to determine the effect of a known degree of hydrophilicity and hydrophobicity of PEO-block-PPO-block-PEO terpolymers on the A549 lung cancer cell as compared with normal human red blood cells (RBC) cells. The block terpolymer has the structure HO(CH₂CH₂O)ₓ(CH₂CH(CH₃)O)_{y}(CH₂CH₂O)ₓH, where x and y are the number of units of EO and PO, respectively. Although the term pluronic is a registered trademark of BASF Aktiengesellschaft Ludwigshafen Germany, it has become used to commonly refer to such terpolyethers. In accordance with the nomenclature established by, BASF the designation L refers to a liquid, P to a paste, and F to a solid at room temperature which is followed by two or three numbers. The first number or first two numbers indicate the approximate molecular weight of the PPO block divided by 300 and the last number refers to the approximate weight percent of the PEO blocks divided by 10. For example F68 refers to a terpolymer of the approximate formula HO(C₂CH₂O)₈₂(CH₂CH(CH₃)O)₃₁(CH₂CH₂O)₈₂H or EO₈₂PO₃₁EO₈₂.

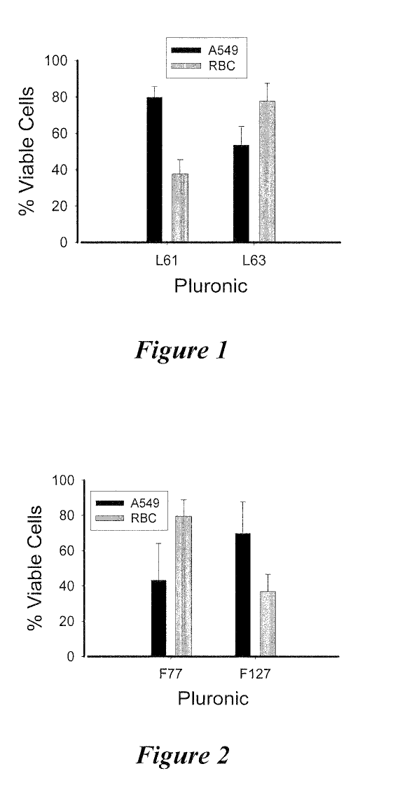

Pluronics having equal sized PPO block but different sized PEO blocks were examined to determine their relative toxicity to A549 and RBC cells. L61 and L63 are approximately EO₂PO₃₁EO₂ and EO₉PO₃₁EO₉, respectively. As can be seen in FIG. 1, L61 displays relatively little toxicity toward A549 cancer cells in vitro where 80% of the cells remaining viable after 100 μL of an 8 mM aqueous solution of the terpolyether was added to a culture of A549, while only 38% of the RBC cells remained viable under these conditions. In contrast, the addition of L63 under the same conditions resulted in only 52% viable A549 cells but 80% viable RBC cells.

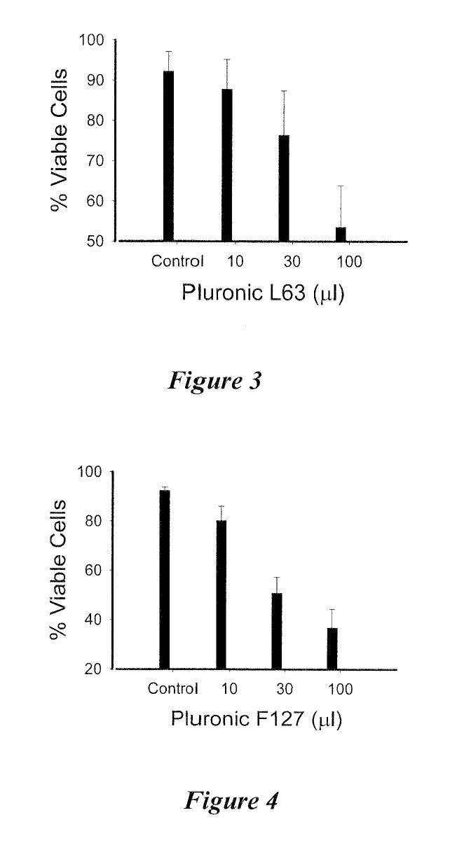

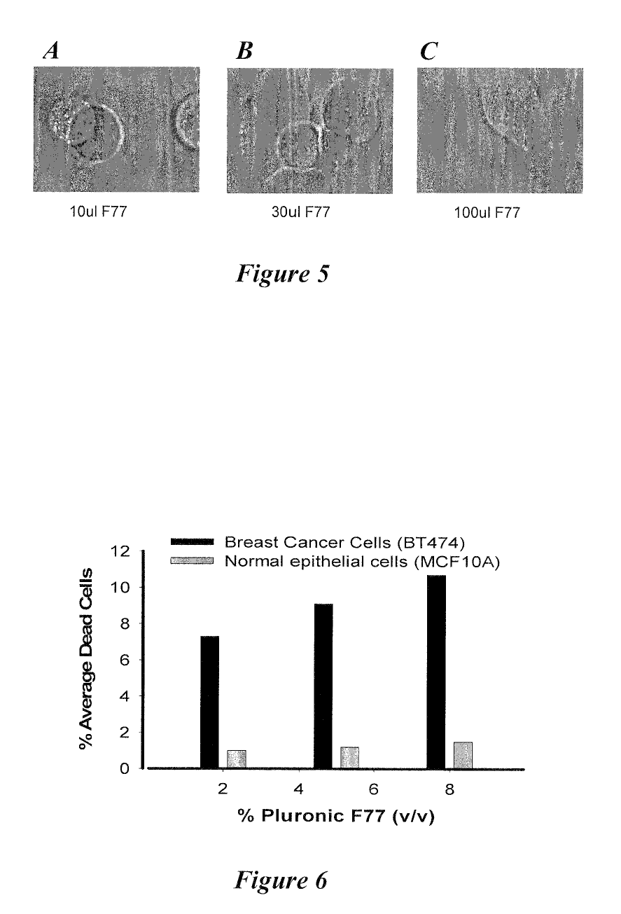

Pluronics having different sized PPO blocks, but equivalent weight percent PEO blocks, were examined to determine their relative toxicity to A549 and RBC cells. F77 and F127 are approximately EO₅₆PO₃₆EO₅₆ and EO₉₅PO₆₂EO₉₅, respectively. As can be seen in FIG. 2, F127 displays relatively little toxicity toward A549 cancer cells in vitro where 72% of the cells remaining viable after 100 μL of an 8 mM aqueous solution of the terpolyether was added to a culture of A549, while only 37% of the RBC cells remained viable under these conditions. In contrast, the addition of F77, under the same conditions, resulted in only 42% viable A549 cells but 80% viable RBC cells. These effects are dose dependant, as shown in FIGS. 3 and 4, and been verified from light microscopy, as shown in FIGS. 5A-5C.

The effects of Pluronic F38, F68 and F77 on Breast cancer cells (BT474) and normal human breast epithelial cells (MCF12A) were evaluated quantitatively using a flow cytometry technique. Cells obtained from the American Type Culture Collection (Manassas, Va.). MCF7 were routinely cultured in 75 cm² culture flasks in Eagle's minimum essential medium (EMEM). The medium was supplemented with L-glutamine (2 mM), sodium bicarbonate (1.5 g/L), sodium pyruvate (1.0 mM), non-essential amino acids (0.1 mM), penicillin-G (50 IL/ml), streptomycine (50 μg/ml), bovine insulin (0.01 mg/ml) and 10% fetal bovine serum (FBS). Breast cancer cells (BT474) were routinely cultured in 75 cm² culture flasks in RPMI 1640 with HEPES buffer (10 mM) where the medium was supplemented with sodium pyruvate (1.0 mM), non-essential amino acids (0.1 mM), penicillin-G (50 IL/ml), streptomycine (50 μg/ml), bovine insulin (0.01 mg/ml) and 10% fetal bovine serum (FBS). Epithelial cells (MCF12A) were routinely cultured in 75 cm² culture flasks in Mammary Epithelial Growth Medium (MEGM) where the medium was supplemented with bovine pituitary extract (50 μg/ml), human recombinant epidermal growth factor (rhEGF) (20 ng/ml), hydrocortisone (0.5 μg/ml), gentamicin sulfate amphoterichin-B (100 μg/ml), bovine insulin (0.01 mg/ml) and cholera toxin (100 ng/ml). All cells were incubated at 37° C. in a humidified atmosphere of 5% carbon dioxide in air.

Quantitative Analysis by Flow Cytometry: Cells (3.0×106/well) were seeded into 6 well tissue culture plates and allowed to adhere for 24 hours. Cells were exposed to Pluronic concentrations e.g. 2% v/v, 5% v/v and 8% v/v for 24 hrs. In each experiment at least 10,000 cells of both cancer BT 474 and normal epithelial cells were used in duplicates. Thereafter, cells were harvested by 0.25% trypsin with EDTA, washed with PBS, centrifuged (at 500 g for 5 min) and resuspended in PBS. Thereafter, 5 microliter of 7-AAD dye was added and held for 5 minutes before carrying out the Flow Cytometry. Pluronic F77 was found to be more efficient than F38 and F68 in selectively killing the BT474 cancer cells, as compared to normal epithelial cells, as shown in FIG. 6. FIG. 6 illustrates that F77 kills BT 474 breast cancer cells substantially higher compared to the killing of normal epithelial cells. Pluronic F38 and F68 showed significantly less effectiveness and selectivity for breast cancer cells over that of normal cells.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method of treating cancer comprising the step of delivering a surface active molecule having a HLB of less than 29 or a mixture of surface active molecules having a HLB of less than 40, and wherein said surface active agent selectively partitions primarily to cancer cells rather than healthy cells.

2. The method of claim 1, wherein said surface active molecule is a non-ionic surfactant having a high hydrophilic fraction and a low hydrophobic fraction.

3. The method of claim 2, wherein said non-ionic surfactant comprises at least one selected from the group consisting of: polyoxyethylene-polyoxypropylene-polyoxyethylene tri-block copolymers; polyoxyethylene sorbitol esters; polyethylene glycol stearates; mixtures of monosterate and distearate esters of mixed macrogols (polyoxyethylene polymer) and free glycol, such as macrogol 15 hydroxystearate; alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction products of polyols and at least one member of the group consisting of fatty acids, glycerides, vegetable oils, and hydrogenated vegetable oils; sugar esters; sugar ethers; sucroglycerides; a reaction product of at least one polyol (glycerol, ethylene glycol, polyethylene glycol, sorbitol, propylene glycol, pentaerythritol, a saccharide) and at least one of a monoglyceride, diglyceride and triglyceride; PEG-10 laurate; PEG-12 laurate; PEG-20 laurate; PEG-32 laurate; PEG-32 dilaurate; PEG-12 oleate; PEG-15 oleate; PEG-20 oleate; PEG-20 dioleate; PEG-32 oleate; PEG-200 oleate; PEG-400 oleate; PEG-15 stearate; PEG-32 distearate; PEG40 stearate; PEG-100 stearate; PEG-20 dilaurate; PEG-32 dioleate; PEG-20 glyceryl laurate; PEG-30 glyceryl laurate; PEG-20 glyceryl stearate; PEG-20 glyceryl oleate; PEG-30 glyceryl oleate; PEG-30 glyceryl laurate; PEG-40 PEG-40 glyceryl laurate; PEG-40 palm kernel oil; PEG-50 hydrogenated castor oil; PEG-40 castor oil; PEG-35 castor oil; PEG-60 castor oil; PEG-40 hydrogenated castor oil; PEG-60 hydrogenated castor oil; PEG-60 corn oil; PEG-6 caprate/caprylate monoglycerides; PEG-6 caprate/caprylate diglycerides; PEG-8 caprate/caprylate monoglycerides; PEG-8 caprate/caprylate diglycerides; polyglyceryl-10 laurate; PEG-40 sorbitan oleate; PEG-80 sorbitan laurate; polysorbate 20; polysorbate 80; POE-9 lauryl ether; POE-23 lauryl ether; POE-10 oleyl ether; POE-20 oleyl ether; POE-20 stearyl ether; tocopheryl PEG-100 succinate; polyglyceryl-10 oleate; Tween 40; Tween 60; sucrose monostearate; sucrose monolaurate; sucrose monopalmitate; PEG 10-100 nonyl phenol series; and PEG 15-100 octyl phenol series.

4. The method of claim 3, wherein said non-ionic surfactant comprises a polyoxyethylene-polyoxypropylene-polyoxyethylene tri-block copolymer.

5. The method of claim 1, wherein said surface active molecule is an ionic surfactant.

6. The method of claim 5, wherein said ionic surfactant comprises at least one of the group consisting of: bile acids and salts, analogues, and derivatives thereof; carnitine fatty acid ester salts; salts of alkylsulfates; salts of fatty acids; sodium docusate; acyl lactylates; mono-acetylated tartaric esters of mono- and diglycerides, diacetylated tartaric acid esters of mono- and diglycerides; succinylated monoglycerides; citric acid esters of mono- and diglycerides; lactylic esters of fatty acids; stearoyl-2-lactylate; stearoyl lactylate; succinylated monoglycerides; mono-acetylated tartaric esters of mono- and diglycerides; diacetylated tartaric acid esters of mono- and diglycerides; citric acid esters of mono- and diglycerides; cholate; taurocholate; glycocholate; deoxycholate; taurodeoxycholate; chenodeoxycholate; glycodeoxycholate; glycochenodeoxycholate; taurochenodeoxycholate; ursodeoxycholate; lithocholate; tauroursodeoxycholate; glycoursodeoxycholate; cholylsarcosine; N-methyl taurocholate; caproate; caprylate; caprate; laurate; myristate; palmitate; oleate; ricinoleate; linoleate; linolenate; stearate; lauryl sulfate; tetraacetyl sulfate; docusate; lauroyl carnitine; palmitoyl carnitine; and myristoyl carnitine.

7. The method of claim 1, wherein said step of delivering comprises: intravenous or intra-arterial delivering; intradermal delivering; injecting into tissue; intraperitoneal delivering; inhaling; intramuscular delivering, applying topically; applying subcutaneously; and oral ingesting.

8. The method of claim 7, wherein said step of delivering is carried out in a dose form or continuously.

9. The method of claim 8, wherein said continuous delivering is carried out using a portable pump.

10. A method of detection and location of cancer cells and tissue comprising the steps of:
providing a surface active agent including a fluorescence moiety, wherein said surface active molecule has a HLB of less than 29 and a relatively high hydrophilic fraction and a relatively low hydrophobic fraction and wherein said surface active agent selectively partitions primarily to cancer cells;
irradiating said surface active agent with electromagnetic radiation to excite said fluorescence moiety; and
observing the fluorescence emission of said fluorescence moiety of said surface active agent, wherein one or more localized volumes of high intensity indicates the presence and location of the cancer cells.

11. The method of claim 10, wherein said fluorescence moiety is derived from at least one of the group consisting of: chlorin e6 or its derivative chlorin e6-Cholin e6-ethylenediamide; polyvinylpyrrolidone (Ce6-PVP); N-acetyl-3,7-dihydroxyphenoxazine or its derivatives; calcein, AM (Glycine, N,N′-[[3′,6′-bis(acetyloxy)-3-oxospiro[isobenzofuran-1(3H), 9′-[9H-]xanthene]-4′,5′-diyl]bis(methylene)]-bis[N-[2-[(acetyloxy)methyoxy]-2-oxoethyl]-, bis[(acetyloxy)methyl]ester) or its derivates; indocyanine green (ICG) dye or its derivatives; 5-(and -6)-Carboxy-2′,7′-dichlorofluorescein; 5-FAM; 6-Carboxyrhodamine 6G; aminocoumarin or rhodamine sulfonated derivatives (e.g.Alexa); 2′-7′-bis(carboxyethyl)-5(6)-carboxyfluorescein derivatives (e.g. BCECF); 4,4-difluoro-3a,4adiaza-s-indacene derivatives (e.g BODIPY FL); Calcein; carboxyfluorescein diacetate (e.g. CFDA); CI-NERF; DTAF; eGFP; eYFP; FDA; FITC; FlAsH; N-Ethoxycarbonylmethyl-6-methoxyquinolinium bromide or derivatives (Fluo3, Fluo4 etc.); Fluorescein and derivatives (e.g. FITC); Fluoro-Emerald; FM 1-43; Magnesium Green; mHoneydew; MitoTracker Green; NeuroTrace 500/525, green fluorescent Nissl stain-RNA; Nissl; Oregon Green 488; PicoGreen dsDNA quantitation reagent; Rhodamine; Sodium Green Na+; SYBR Green I; SYTO 13-DNA; TO-PRO-1; and TOTO-1-DNA.
